(19) United States
(12) Patent Application Publication
(10) Pub. No.: US 2003/0186311 A1
(43) Pub. Date: Oct. 2, 2003

(54) Parallel analysis of molecular interactions

(75) Inventors: Eric Henderson, Ames, IA (US); James Johnson, St. Charles, IA (US); Saju Nettikadan, Ames, IA (US)

Correspondence Address: Jill A. Fahrlander, Michael Best & Friedrich LLP, One South Pinckney Street, P.O. Box 1806, Madison, WI, 53701-1806, US

(73) Assignee: BioForce Nanosciences, Inc., Ames, IA

(21) Appl. No.: 10/427,003

(22) Filed: Apr. 30, 2003

Related U.S. Application Data
(63) Continuation-in-part of application No. 10/225,080, filed on Aug. 21, 2002.
Application No. 10/225,080 is a continuation of application No. 09/745,362, filed on Dec. 21, 2000, now abandoned.
Application No. 09/745,362 is a division of application No. 09/574,519, filed on May 18, 2000, now Pat. No. 6,573,369.
(63) Continuation-in-part of application No. 09/974,757, filed on Oct. 9, 2001.
(60) Provisional application No. 60/135,290, filed on May 21, 1999.
(60) Provisional application No. 60/238,556, filed on Oct. 10, 2000.

(51) Int. Cl. C12Q 1/68; G01N 33/53

(52) U.S. Cl. 435/6; 435/7.1

(57) ABSTRACT

Provided are methods of detecting molecular interactions using arrays and near field scanning probe techniques. Also provided are methods of characterizing binding interactions under defined reaction parameters, methods of determining antibody binding specificity, methods of selecting a substrate for an array of immobilized molecules and methods of determining molecular occupancy time with respect to binding interactions.

### CROSS REFERENCE TO RELATED APPLICATIONS

This application is a Continuation-in-Part of prior application Ser. No. 10/225,080, filed Aug. 21, 2002, which is a Continuation of U.S. application Ser. No. 09/745,362, filed on Dec. 21, 2000 which is a Division of application Ser. No. 09/574,519, filed on May 18, 2000 which claims priority to Application No. 60/135,290, filed on May 21, 1999. This application is also a Continuation-in-Part of U.S. application Ser. No. 09/974,757, filed Oct. 9, 2001 which claims priority to Application No. 60/238,556, filed on Oct. 10, 2000.

### FIELD OF THE INVENTION

The present invention relates to methods of detecting and characterizing molecular binding interactions using arrays. The invention also relates to analysis of arrays using near field scanning probe techniques.

### INTRODUCTION

A variety of analytical techniques are conventionally used to characterize molecules and molecular interactions in the laboratory. Because monoclonal antibodies recognize a single antigenic determinant, or epitope, they have become widely relied upon by investigators seeking to elucidate the nature of complex molecular entities and events. For example, antibodies are routinely employed in enzyme-linked immunosorbent assays (ELISA), immunofluorescence assays, capture immunoassays, agglutination assays and western blot assays, as well as other commonly employed laboratory assays and techniques. Similarly, non-antibody affinity entities such as peptide and nucleic acid aptamers also have the ability to bind to specific target molecules and, therefore, well-defined assay methodology is beneficial for research applications.

It is often necessary to evaluate and characterize antibodies and aptamers prior to their use as research tools in order to define, among other things, epitope binding specificity and binding properties, suitability of the antibody or aptamer for immobilization on a solid surface, and conditions under which optimum binding occurs.

Many techniques exist for the evaluation of antibodies interacting with a soluble and/or particulate antigen. One is Immuno Electron Microscopy (IEM) and its variant, solid phase immuno electron microscopy (SPIEM). These techniques are deficient in that they cannot assess the avidity or affinity of the antibody-antigen interaction without processing data from numerous experiments. IEM and SPIEM are exceptionally incompatible with evaluation of many antibodies, as they require much manual manipulation on individual samples and are, therefore, extremely difficult to perform in a highly parallel format.

Yet another method is capture immunoassay, i.e., capture EIA and its cognates, which is performed on a modified plastic or retentive paper such as nitrocellulose, wherein capture of the antigen by the antibody is recognized by a secondary antibody conjugated to an enzyme that effects conversion of a substrate to a product. This process is insensitive. Broadly interactive antibodies may cause a positive reaction and neither quantitative nor qualitative assessment of binding affinities are easily obtained.

### SUMMARY OF THE INVENTION

The present invention encompasses, among other things, methods of rapidly characterizing antibodies and other affinity molecules with respect to epitope specificity and binding characteristics in a parallel format. The methods described herein do not require a secondary antibody or other label, and do not require additional steps such as photodetection or development of a chromogenic substrate. Because antibodies, as proteins, are sensitive to environmental conditions, the methods can be carried out under varying conditions or in solution.

In a first aspect, the invention provides a method of detecting a molecular interaction. The method comprises steps of contacting an array with one or more target molecules, interrogating the array with a probe having a tip to create a profile of the array, and evaluating the profile to detect an interaction between at least one affinity molecule and at least one target molecule. In this method, the array comprises a plurality of different affinity molecules in discrete domains, and each domain has a predefined address in the array.

In another aspect, the invention provides a method of determining antibody specificity. The method comprises steps of contacting an antibody array with an antigen, interrogating the array with a probe having a tip to create a profile of the array, evaluating the profile to detect an antibody-antigen interaction in one or more of the domains, and correlating the antibody-antigen interaction with antibody specificity. The invention also provides a method of determining antibody specificity performed by contacting an antigen array with antibodies.

In yet another aspect, the invention provides a method of characterizing a molecular interaction. The method comprises steps of contacting an array with one or more target molecules under defined reaction parameters, interrogating the array with a probe having a tip to create a profile of the array, evaluating the profile to detect an interaction between at least one affinity molecule and at least one target molecule in one or more domains, and correlating the interaction with the binding conditions to characterize the molecular interaction. In this method, the array comprises a plurality of affinity molecules in discrete domains, and each domain has a predefined address in the array.

In still another aspect, the invention provides a method of selecting a substrate for an array of immobilized molecules. The method steps comprise contacting an array with at least one target molecule, interrogating the array with a probe having a tip to create a profile of the array, evaluating the profile to detect a molecular interaction in one or more of the domains, and selecting one or more of the substrates based on the profile. In this method, the array comprises a plurality of substrates arranged in discrete domains and at least one affinity molecule disposed on the substrates in each of the domains.

In still another aspect, the invention provides a method of determining target occupancy time. The method comprises contacting an array with one or more target molecules, interrogating the array with a probe having a tip to detect onset of binding between at least one target molecule and at least one affinity molecule, interrogating the array with a probe having a tip to detect dissociation of at least one target molecule and at least one affinity molecule, and measuring the time between onset of binding and dissociation to determine target occupancy time. In this method, the array comprises a plurality of affinity molecules in discrete domains, each domain having a predefined address in the array.

### BRIEF DESCRIPTION OF THE FIGURES

FIGS. 1A and 1B are schematic drawings depicting embodiments of a method of detecting a molecular interaction in accordance with the present invention

FIG. 2 is a schematic drawing depicting one embodiment of determining antibody specificity in accordance with the present invention.

FIGS. 3A and 3B are schematic drawings depicting a further embodiment of determining antibody specificity in accordance with the present invention.

FIGS. 4A and 4B are schematic drawings depicting a further embodiment of determining antibody specificity in accordance with the present invention.

FIG. 5 is a schematic drawing depicting a further embodiment of determining antibody specificity in accordance with the present invention.

FIG. 6 is a schematic drawing depicting an embodiment of a method of selecting a substrate in accordance with the present invention.

FIG. 7 shows AFM images of three monolayers of different commercial antibodies (panels A, B and C) bound to their target antigen, bacteriophage fd. Panels B and C are contrast-enhanced to facilitate data interpretation.

FIG. 8 shows AFM images and corresponding height reference profiles for anti-HIV gp120 antibody bound to viral protein in a nanoarray format.

### DETAILED DESCRIPTION OF SEVERAL EMBODIMENTS

In the presently described invention, the combination of molecular array technology and near field proximal probe microscopy provides a valuable tool for rapid screening of molecular interactions. Specifically, the presently described methods provide a means for rapid, high throughput analysis of affinity molecule-target molecule interactions, including antibody-antigen interactions, and further provides a tool for determining specific binding domains and evaluating binding kinetics, e.g., affinity constants. The method also allows for rapid determinations of suitable binding conditions, including substrate selection. The molecules used in the described methods may, optionally, be label-free, that is, there is no requirement for a fluorescent, radioactive, enzymatic or other molecular “tag.” Moreover, methods in accordance with the invention can be performed in any environment, including ambient air, gas phases, aqueous phases, or solutions. The environment can include components that do not participate in the molecular interaction of interest.

Detection of Molecular Interactions

As used herein, a “molecular interaction” refers broadly to an affinity molecule-target molecule interaction. Non-limiting classes of molecular interactions include antibody-antigen, enzyme-substrate, aptamer-target and ligand-receptor interactions. Examples of particular molecular interactions that may be detected in accordance with the invention include nucleic acid-nucleic acid, protein-nucleic acid, protein-protein and lipid-protein interactions.

“Interaction,” as used herein, refers broadly to, e.g., binding, effecting a conformational change, cleaving, polymerizing, catalyzing, phosphorylating, glycosylating, acetylating and farnesylating. Suitably, the interaction is a binding interaction between two or more molecules.

“Binding,” as used herein and in the art, refers to any of covalent, non-covalent, electrostatic, Van Der Waals, ionic and hydrophobic binding, and may be specific or non-specific. In many suitable embodiments of the present method, binding is specific.

As used herein, an “affinity molecule” is any natural or synthetic peptide or oligonucleotide species immobilized on a substrate that is capable of binding a target molecule. Non-limiting examples of affinity molecules include antibodies or portions thereof, aptamers and receptors. As will be appreciated by those of skill in the art, an affinity molecule can also be an antigen when the target molecule is an antibody.

Accordingly, a “target molecule” is any peptide, oligonucleotide, lipid, carbohydrate, glycoprotein or chemical species capable of binding to an affinity molecule. A typical target molecule is an antigen, which may comprise any of the aforementioned molecular species. An “antigen,” as used herein, is any molecular species that binds an antibody, or any portion thereof. The definition of “antigen” used herein expressly does not require that such a molecular species have any particular effect with respect to the immune system of any living subject. A target molecule can also be an antibody, i.e., when the immobilized species is an antigen of interest. As will also be understood by those of skill in the art, an antibody can itself be considered a target for another antibody (e.g., rabbit anti-goat antibody). Targets in a liquid sample may be known or unknown. In other words, methods conducted in accordance with the present invention may be used to detect the presence of a target in a sample, or may be used to characterize a known binding interaction.

An “aptamer” is a small molecule affinity reagent that is randomly generated or rationally designed to bind a particular target of interest. Aptamers may be short oligonucleotides (see, e.g., Brody E N and Gold L, “Aptamers as therapeutic and diagnostic agents,” Reviews in Molecular Biotechnology 74: 5-13 (2000); Macaya R F et al., “Thrombin-binding DNA aptamer forms a unimolecular quadruplex structure in solution,” Proc. Natl. Acad. Sci. 90: 3745-3749 (April 1993)), or peptides (see, e.g., Colas P et al., “Genetic selection of peptide aptamers that recognize and inhibit cyclin-dependent kinase 2,” Nature, 380: 548-550 (April 1996). Peptide aptamers may also refer to peptide sequences engineered into a larger protein scaffold.

An “array” refers to a plurality of spatially arranged domains disposed in known locations, or “addresses,” on a suitable substrate. Suitable substrates include gold, quartz, mica, glass, silicon, chromium, filter matrices (e.g., nitrocellulose or nylon) and plastic (e.g., polystyrene). Suitable substrates, in accordance with the invention, are not limited to any particular surface roughness, however, surface roughness should be controlled such that molecular imaging is not hindered. An array, as used herein, can be a “nanoarray,” which has domain areas of about 50 square nanometers to about one square micron, or can be a “microarray” having larger domains, up to and including about 200 square micrometers. Arrays used in the present methods are substantially planar. As used herein and in the art, “substantially planar” refers to a generally two-dimensional surface on which domains are created. However, as will be immediately understood, the molecules immobilized in the domains of the array (defined below), may extend from the plane of the substrate surface in three dimensional space.

A “domain” or “molecular domain” or “affinity domain” is a discrete region of immobilized species wherein the individual molecules within a single domain are of the same species. Suitably, the domain areas are about 50 square nanometers to about 1 square micrometers. In some embodiments, each domain contains a plurality of affinity molecules. As will be understood, the number of molecules deposited in each domain will be dependent on the size of the molecules and the size of the domains, as determined by the particular user-defined application. In some embodiments, molecules of neighboring domains are of different species. “Different” as the term is used herein to describe molecular species, means that a detectable variation exists between two or more molecules being compared. For example, two molecules having non-identical sequences would be different, as would two molecules having non-identical post-translational modifications. Similarly, a library of antibodies raised against a particular antigen, but which bind different epitopes (e.g., are derived from different hybridomas) are different. “Plurality,” as used herein, refers to two or more.

Suitable methods of creating arrays of affinity molecules are described in co-pending application Ser. No. 09/929,865 entitled “Nanoscale Molecular Arrayer,” incorporated herein by reference in its entirety. Other suitable methods of creating arrays are described in U.S. Pat. No. 6,146,899 to Porter, U.S. Pat. No. 5,837,832 to Chee and U.S. Pat. No. 6,110,426 to Shalon, each of which are also incorporated herein by reference. Using these and other arraying methods known in the art, affinity molecules can be attached to an array substrate in discrete domains via a number of suitable chemical or biological tethering techniques.

Typically, affinity molecules are placed in contact with a prepared substrate surface and allowed to spontaneously adsorb onto the surface. Alternatively, chemical tethering methods are suitably carried out by modifying the substrate surface in each of the domains to facilitate covalent attachment. Non-limiting examples of suitable surface modifications include those that provide carbodiimide, succinimide or malimide groups. A “spacer” can be added to an affinity molecule prior to its immobilization to improve its reactivity with its target. Typical spacers include polyethylene glycol and alkanethiolates in which the alkane chain has about 12 to about 18 carbons. A suitable attachment method is described in, e.g., U.S. Pat. No. 6,518,168 to Clem et al., incorporated herein by reference.

Biological tethering can be accomplished by coating a surface with streptavidin and contacting with biotin-modifyied antibody or aptamer. Another suitable method of biological tethering is modifying the substrate surface with protein G or protein A, each of which binds the F_{c} region of an antibody. This method suitably orients the antibodies such that the hypervariable, or epitope binding, regions are directed away from the surface and are therefore free to bind their target.

As will be appreciated by those of skill in the art, the use of aptamers, which are designed and synthesized de novo, provides the opportunity to “engineer” any of the aforementioned chemical or biological tethers into the molecule in precisely designated locations in the molecule.

Near Field Probe microscopy is suitably used to interrogate the arrays in the methods of the invention. Near Field Probe microscopy encompasses a family of instruments called scanning probe microscopes. One member of this family, the Atomic Force microscope (“AFM”), has become widely accepted in a variety of fields and is suitable for use in the present invention. Briefly, in atomic force microscopy, a microcantilever probe having a sharp tip is scanned over a surface using piezoelectric control mechanisms. Typically, the interaction of the probe with the surface is recorded and reported via an imaging system operably connected to the AFM. Other near field instruments suitable for use in the present invention include near field scanning optical microscopes and scanning tunneling microscopes. Each of these instruments is capable of detecting changes in topography, force, heat, electromagnetic properties, resonance frequency or other physical properties that can be correlated with interaction between affinity molecules and target molecules disposed on the array.

Accordingly, the term “interrogating an array” refers to scanning the array with a probe having a tip. In some embodiments, the probe is a microcantilever. In some cases, the AFM probe contacts the molecules in the array directly and the amount of force applied to the surface can be calculated based on the known spring constant of the microcantilever and the amount of deflection. By scanning the topography of the surface of the array, the direct contact of the probe provides height information, which can be a reliable indicator of molecular binding. In other cases, an array may be interrogated indirectly e.g., when resonance frequency of a single molecule or affinity-target pair is measured, the change in frequency of a rapidly vibrating cantilever as it approaches the sample can be determined.

In further embodiments, a molecule, i.e., a target molecule or an affinity molecule, may be disposed on a microcantilever probe tip. This orientation allows for determinations of physical properties or forces related to binding or unbinding interactions. This is accomplished by measuring binding force, or rupture force, as described more fully herein below. Other physical properties suitably measured by scanning probe techniques in methods of the present invention include friction, adhesion, viscoelasticity and compliability. These properties are all measured by determining mechanical effects exerted on the scanning probe (e.g., twisting, bending, oscillating, resonating, phase shifting).

“Contacting an array,” as the term is used herein, refers to the delivery of target molecules to the domains of the array. Delivery of a liquid sample containing target molecules is suitably accomplished by using a flow cell, by deposition in each of the domains with a probe (e.g., an AFM probe), pipette or micropipette, by utilizing microfluidic delivery devices known to those of skill in the art, by dipping or floating the arrays in liquid samples, or by any method suitable for bringing the affinity and target molecules in contact such that a molecular interaction can occur. In the case of probe transfer (using, e.g., a microcantilever or nanocantilever) the volume of material used can be nanoliters or less, thereby conserving the target material and providing a means for delivery of variants of the target material to the same array, if desired. In some cases, humidity is suitably controlled in the environment surrounding the array and probe instrument to prevent sample loss. Suitably, a chamber can be included to maximize sample contact and minimize evaporation.

In other embodiments of the invention, the target molecules are disposed on a probe tip and brought into contact with the affinity molecules in the array via piezoelectric control of the microcantilever in the x, y and z directions. It is to be understood that in this embodiment, contacting the array with one or more target molecules is accomplished simultaneously with interrogating the array. This embodiment of the invention is particularly suitable for determinations of reaction kinetics or other characterizations of the interaction, as described below.

A “profile” as the term is used herein, refers to the data set of information acquired by the interrogation process. Accordingly, “evaluating” a profile is processing information provided by the profile regarding, e.g., whether a binding interaction has taken place in any of the domains. Thus, for a topographic data set, the profile of the surface would correspond to the topographic information at each point on the surface for which data is gathered. This profile would be examined and the topographic data correlated with the occurrence or non-occurrence of a binding event. Similarly, for a force measurement data set, the profile would include a force value determined at each point at which data is acquired. Again, this would be incorporated into a complete data set or force profile. It is noteworthy that different types of data (e.g., force and topography) can be accumulated at the same time and displayed as complex (e.g., differently color coded and overlapping) profiles to enhance the data interpretation process.

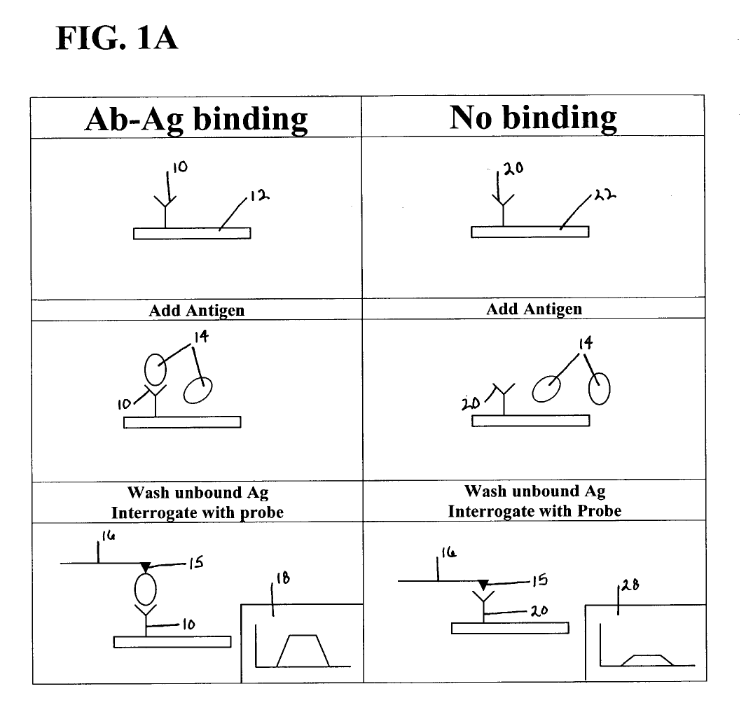

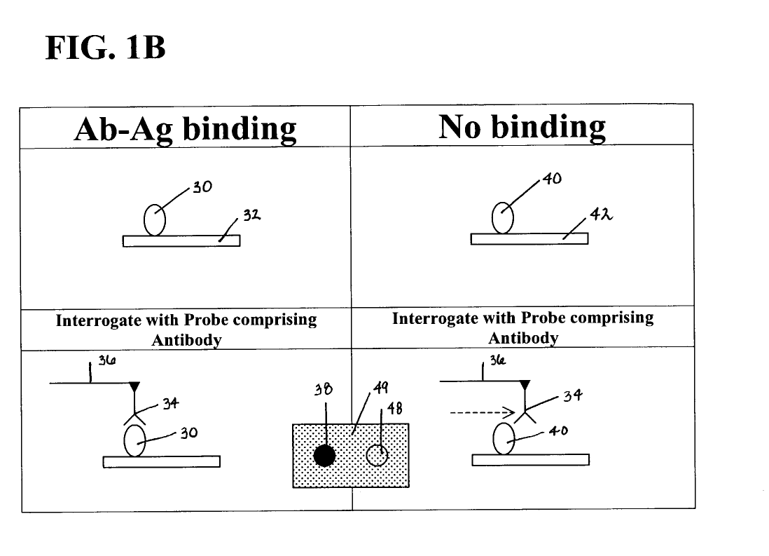

A non-limiting embodiment of the method of detecting a molecular interaction is shown in FIGS. 1A and 1B. For ease of reference, the schematic diagrams of domain 12 and domain 22 each have a single antibody 10, 20 disposed thereon, although domains may suitably comprise a plurality of affinity molecules. Domains 12 and 22 are contacted with a sample containing antigen molecules 14. As shown, antibody 10 binds a single molecule of antigen 14, whereas antibody 20 does not bind antigen 14. After a wash step removes unbound antigen from the domains, a surface probe 16 with a tip 15 is used to interrogate the topography of the domains. The resultant profile 18 of domain 12 containing antibody 10 bound to target antigen 14 shows increased height relative to the profile 28 that results from a scan of domain 22 containing antibody 20.

An alternative embodiment is depicted in FIG. 1B. Here, domain 32 contains antigen 30 and domain 42 contains antigen 40. Interrogation with a probe having a tip comprising antibody 34, which binds antigen 30, but not antigen 40, results in, for example, a force map profile 49 wherein a positive signal 38 corresponds to the scan of domain 32, and a negative signal (e.g., similar to background) corresponds to the scan of domain 42. In this example, a positive signal 38 for domain 32 is representative of the increase in force required to advance probe 16 in the x-y direction or lift the probe in the z direction. Alternatively, measurements of friction, viscoelasticity binding force, rupture force, affinity and avidity are suitably made and suitably presented in a profile.

Determining Antibody Specificity

The methods of the invention are suitably used in the determination of antibody specificity. For example, the methods described herein are suitably used to provide a means of “cataloguing” or “typing” antibodies in a population known to bind a particular antigen, e.g., products of a hybridoma library. The methods in accordance with the present invention enable the researcher to quickly and accurately evaluate antibody products of hybridomas for specific characteristics desirable in various forms of immunodetection assays. This applies in particular to categories of immunoglobins which have previously been difficult to characterize in detail, i.e. those that interact with particulate antigens such as viruses, recombinant particles produced from genetically engineered organisms, bacteria, and sub-cellular particulate components from prokaryotic and eukaryotic organisms. These classes of interactions are easily detectable in accordance with the present invention.

In some instances, a “pre-screen,” such as an ELISA assay, western blot or immunoprecipitation assay, is optionally used to determine antigen binding capability of antibodies in a population. Although such a pre-screening step is not necessary, it may be useful in selecting antibodies for further screening by methods described herein.

Some embodiments of the present method require that the antigen be “modified.” As used herein, a “modified antigen” is one in which one or more of its native epitopes are unavailable to bind to an antibody capable of binding the unmodified antigen. Suitably, an antigen may be modified by binding with blocking antibodies or affinity molecules of known specificity, or by substitution or deletion mutagenesis. Suitable techniques for mutagenizing an antigen are well known to those of skill in the art.

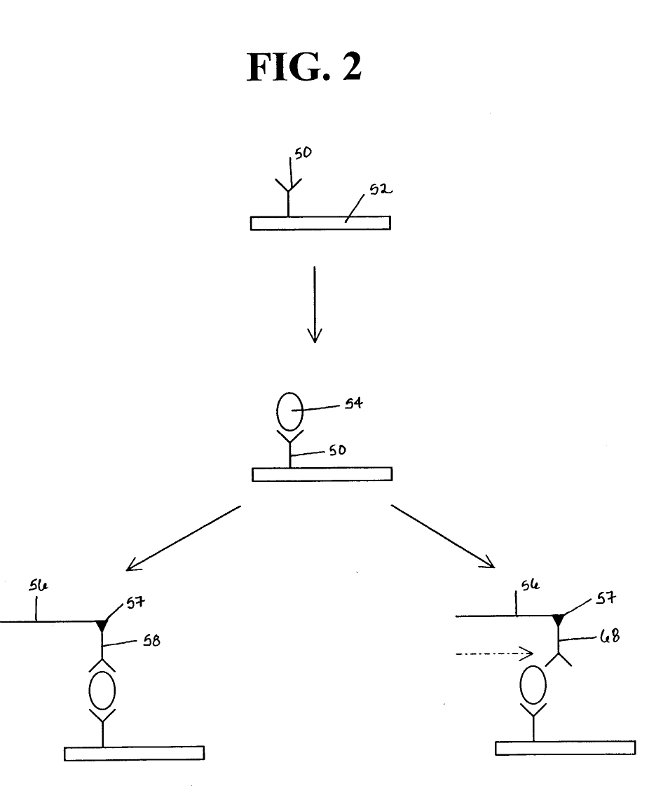

One embodiment of the presently described method is schematically represented in FIG. 2. The specificity of immobilized antibody 50 in domain 52 is determined by first contacting domain 52 with soluble antigen 54. Next, probe 56 having a tip 57 comprising antibody 58 or 68, each of which has known specificity for different epitopes of antigen 54 (as determined by available methods known to those of skill in the art) is used to interrogate the immobilized antibody 50-antigen 54 pair. If the immobilized antibody 50 has different epitope binding specificity than antibody 58, the epitope for antibody 58 will be free and interrogation of domain 52 will result in binding of antibody 58 to its corresponding epitope on antigen 54. Evaluation of the resultant profile will therefore reveal that the immobilized antibody 50 and the tip-bound antibody 58 bind different epitopes of antigen 54. If, however, the immobilized antibody 50 binds the same epitope as the tip-bound antibody 68, the epitope for antibody 68 will be occupied by immobilized antibody 50 and interrogation of domain 52 will not result in binding of tip-bound antibody 68. Upon evaluation of the resultant binding/unbinding force profile, it is determined that either the immobilized antibody 50 has the same specificity as antibody 68, or the binding of antibody 50 to its epitope sterically hinders the binding of antibody 68 to its epitope, e.g., the epitopes overlap.

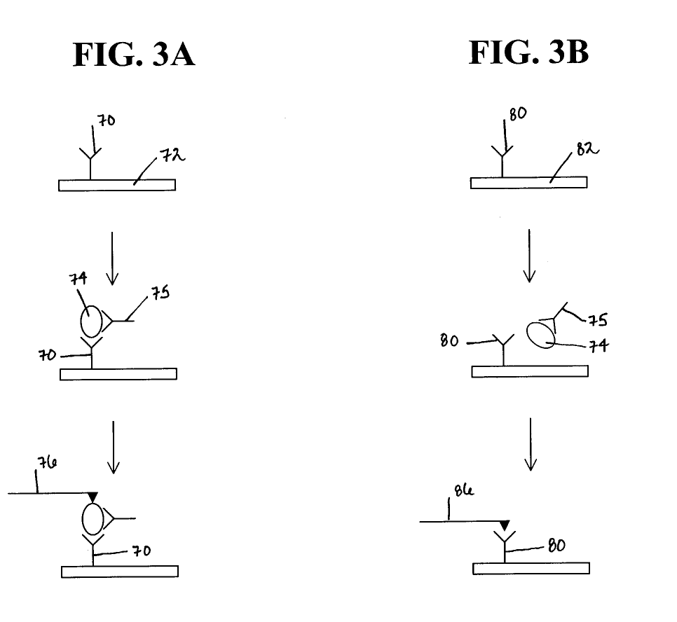

FIGS. 3A and 3B depict a further approach to determining epitope specificity in accordance with the present invention. Domains 72, 82 containing antibodies 70, 80 of unknown epitope specificity are contacted with target antigen 74. “Blocking” antibody 75 of known epitope specificity is introduced either by preincubation with antigen 74 prior to contacting the domains 72, 82 of the array, or can be introduced as a soluble factor in subsequent step. In FIG. 3A, unknown antibody 70 binds a different epitope of antigen 74 than antigen 75. Therefore, interrogation with a probe 76 having a tip will result in an increased height profile of domain 72. In FIG. 3B, unknown antibody 80 binds, or at least overlaps or is proximate to, the epitope on antigen 74 bound by antibody 75. Therefore, interrogation with a probe 86 having a tip will not result in an increased height profile.

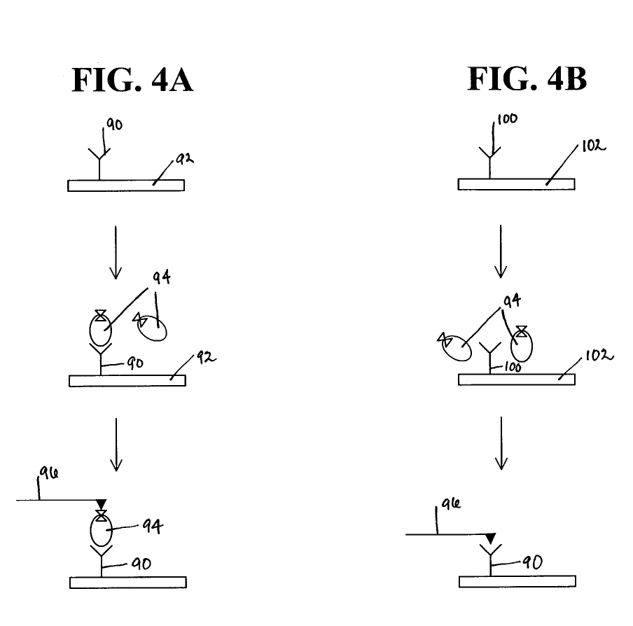

In yet another approach to epitope mapping, the target antigen can be modified at the molecular level, thereby changing its epitope characteristics. For example, if the target molecule is a protein for which the coding sequence is known, modifications, e.g. mutations, of the sequence can be induced in a rational or random fashion and the modified sequence expressed to generate modified target molecules. These modified proteins, e.g., antigens, can then be used in the AFM screening technique described herein to determine specificity of antibodies that bind to the unmodified antigen. For example, as shown in FIGS. 4A and 4B, antigen 94 has a deleted epitope (depicted by an “x”). Antibody 90 binds an epitope other than the deleted epitope, and therefore, a surface probe scan of domain 92 will show increased height. On the other hand, antibody 100 is specific for the deleted epitope and thus, unable to bind. A surface probe scan of domain 102 will not show an increase in its height profile.

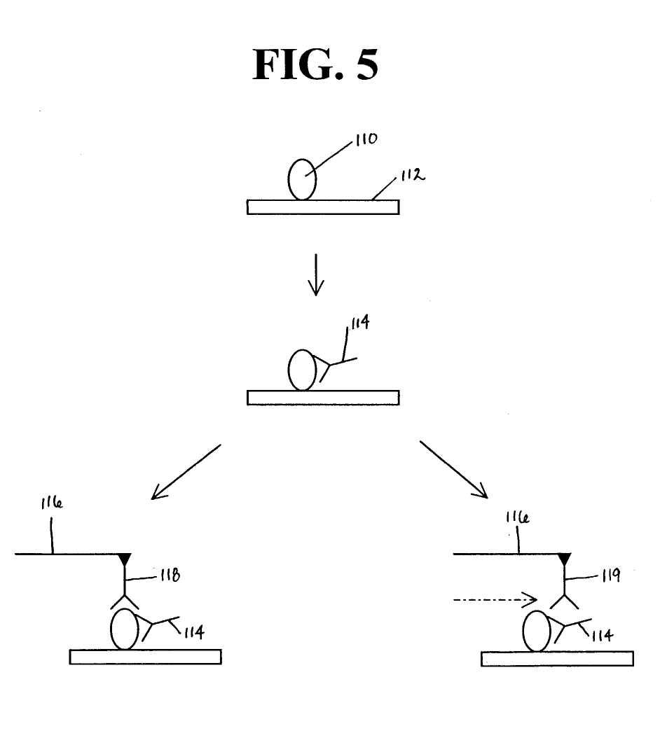

The methods of determining antibody specificity in accordance with the present invention can also suitably be carried out using an antigen array. As depicted in FIG. 5, antigen 110, disposed in domain 112, is contacted with a sample containing antibody 114 of unknown specificity, which binds to antigen 110. Probe 116 having a tip comprising an antibody 118 of known specificity is used to interrogate domain 112. Antibody 118 binds an epitope other than that of antibody 114, and therefore, it can be determined from the profile that antibody 114 is not directed against the same epitope as that against which antibody 118 is directed. If tip-bound antibody 119 is directed to an identical (or overlapping or proximate) epitope as that to which antibody 114 is directed, however, the profile will reveal that no binding interaction occurred between the tip-bound antibody and immobilized antigen 110.

Characterization of Molecular Interactions

The methods described herein provide a means of characterizing interactions between binding partners, e.g., antibody and antigen pairings. As will be appreciated, interactions may be characterized with respect to a single intermolecular pairing, or may be characterized and expressed with respect to a population of molecules. In accordance with the present invention, a surface probe can be used to measure and calculate a number of parameters, including friction, binding force, affinity, avidity and rupture force. “Friction” as the term is used herein, refers to the adhesion between two entities as they pass each other in close proximity. “Binding force,” as the term is used herein, refers to the force equivalent of the energy absorbed or released upon binding of two molecules. “Affinity” as the term is used herein, refers to the strength of the bond between two or more molecules, i.e., the attractive force or energy between molecules. In some embodiments, affinity can be expressed as a ratio of the number of bound/unbound molecules in a population of molecules at steady state. “Avidity,” as the term is used herein, refers to the functional affinity between two or more molecules, whose interaction is strengthened by multiple contact points.

“Rupture force” refers to the force required to reverse, i.e., “break” a molecular interaction between two or more bound molecules. Each of these binding characteristics can be measured as a change in voltage on a photodiode, which in turn is caused by the degree of cantilever deflection (generally in the z direction) or torsion (generally in the x and y directions) during interrogation of an array.

Moreover, the presently described methods can be used to determine characteristics of binding interactions relative to defined reaction parameters. As used herein, “defined reaction parameters” refers to user-defined reaction conditions, i.e., user control of the environment of the binding interaction. “Defined” in the context of the invention can refer to known reaction parameters or unknown components in a reaction medium, i.e., it can be determined whether a molecular interaction proceeds in the presence of known or unknown soluble or particulate species present in the reaction solution. For example, binding between an antibody and an antigen can be evaluated in serum or other biological fluids. Non-limiting examples of reaction parameters that can be controlled by the user include tonicity, pH, humidity, temperature and pressure. In addition, the user may evaluate stability of a prepared array using this method. Thus, the invention allows for the selection of affinity molecules that have the capability to bind their target under specific conditions. In particular, the presently described embodiment of the invention provides biological materials that are tailored for use under conditions to which an array of affinity molecules will be exposed. The method is particularly useful in complex analyses where only marginally compatible processes must be integrated.

Selection of Substrate

Similarly, detection of a particular binding interaction according to the invention also provides a means for selection of substrate. This is because the particular method/substrate used to immobilize affinity molecules is immediately characterized upon binding, i.e., it can be determined whether the immobilization technique and/or substrate is suitable for the affinity molecules under consideration. Therefore, the presently described methods provide a means for selecting a substrate for an array of immobilized molecules.

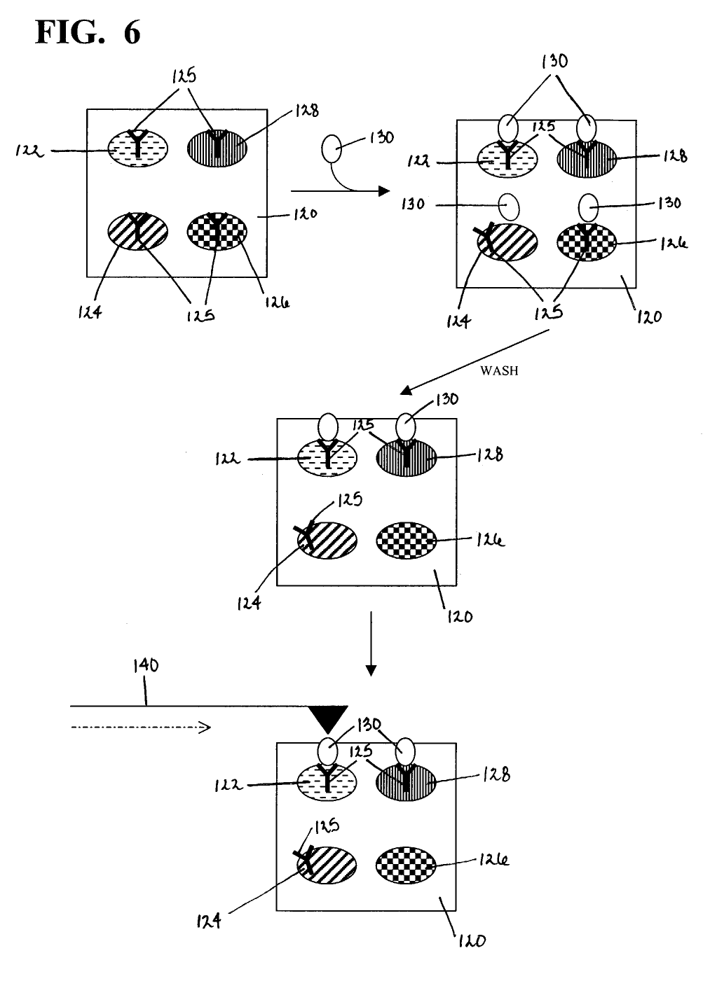

As schematically depicted in FIG. 6, an array 120 of different substrates 122, 124, 126, 128 can be evaluated for ability to immobilize functional antibody 125. The array contacts antigen 130 for a sufficient period of time to allow binding to occur. A wash step can optionally be used to remove all unbound antigen and all non-immobilized antibodies. Interrogation with a probe 140 having a tip, as described above, provides a binding profile which reveals that neither substrate 126 nor substrate 124 is suitable for the antibody-antigen interaction evaluated.

Determination of Target Occupancy Time

The presently described methods provide a means for determining target occupancy time. As used herein, “target occupancy time” refers to a measurement of the length of a time a target molecule is bound to its corresponding affinity molecule at equilibrium.

A surface probe scanning technique is used to measure target occupancy time by scanning an array of affinity molecules that has been contacted with putative target molecules. As described above, target molecules can contact the array either in a liquid sample or tethered to the probe tip. Immediately, or as soon as possible, after delivery of the target molecules to the array, the array is interrogated with a probe having a tip to detect onset of binding. Suitably, interrogation may be based on topography, force or other known interrogation techniques known in the art. As used herein, “onset of binding” refers to the initiation of a binding interaction in one or more domains of the array, as detected according to the interrogation methods described herein above.

After the onset of binding is detected in one or more domains, the array is interrogated at intervals, which can be regular or random, with a probe having a tip until dissociation of a previously bound affinity molecule is detected. As used herein, “dissociation” refers to the release of a target molecule from its corresponding binding site on an affinity molecule.

The occupancy time determined by the present method can represent an average time measured in multiple domains, or can represent an average for a single domain containing a plurality of affinity molecules. Alternatively, the occupancy time can be measured for a single molecular pair.

As will be understood by those of skill in the art, the present method will be useful in providing occupancy time determinations for enzyme/substrate interactions, antibody/antigen interactions and receptor/ligand interactions, as well as other molecular pairings.

### EXAMPLES

The following examples are provided to assist in a further understanding of the invention. The particular materials and conditions employed are intended to be further illustrative of the invention and are not limiting upon the reasonable scope thereof.

### Example 1

### Hybridoma Screening

A large pool of monoclonal antibodies specific for interferon-gamma (IFN-γ) is created using hybridoma technology and the pool is pre-screened using a standard ELISA protocol for those antibodies that are optimal for further immunoassay development.

a. Monoclonal Antibody Array Development

Antibodies reactive in the ELISA pre-screen are deposited in 30 μm diameter spots in discrete domains on a gold array surface using a microjet device. The antibodies then are allowed to spontaneously attach to the gold surface. Multiple arrays are produced.

b. Characterization of Monoclonal Antibodies Using Blocking Antibodies

A series of “blocking” antibodies of known binding specificity are added to a pure preparation of IFN-γ in buffer such that the corresponding binding sites on IFN-γ are completely occupied, i.e., at saturation. After incubating for 30 minutes, the blocking antibody/IFN-γ mixtures are serially added to the antibody arrays of Example 1a and incubated for 30 minutes, rinsed three times with PBS, and imaged by AFM.

As the blocking antibody of known specificity binds or sterically inhibits the corresponding binding site of IFN-γ, it is expected that a subpopulation of antibodies in the array will bind to IFN-γ at one of the remaining available IFN-γ binding sites. As further experiments are carried out on identical arrays using other blocking Ab/IFN-γ, it is determined that another subpopulation will bind IFN-γ at another of the remaining sites. From these experiments, it can be determined that antibodies in the array that bind “blocked” IFN have specificity for one of binding sites other than those of the blocking antibodies. After performing the experiment using differentially blocked IFN-γ, binding specificity of each of the arrayed antibodies is determined.

The site specificity of the monoclonal antibodies can be confirmed and further characterized using deletion mutants as described below in Example 1c.

c. Characterization of Monoclonal Antibodies Using Deletion Mutagenesis

In a further approach, IFN-γ deletion mutants lacking contiguous amino acid segments of 1-25 amino acids are produced using standard recombinant techniques to remove putative binding domains while maintaining the correct reading frame. Alternatively, peptides of known amino acid sequences can be synthesized using well-known techniques to produce synthetic deletion mutants.

The recombinant or synthetic IFN-γ mutants are delivered to the array and allowed to bind, followed by AFM imaging. A population of antibodies in the array will bind to the native IFN-γ protein, while failing to bind one or more mutants having a deleted sequence. It can be inferred from the experimental results that the deleted sequence contains, or at least overlaps, the binding domain specific for the non-reactive antibodies.

### Example 2

### Aptamer Characterization

Aptamers of 15 amino acids having a high binding affinity to the F_{c} region of an IgG molecule are characterized as described below.

a. Initial Screening

The initial isolation and amplification process for the F_{c}-binding aptamers was carried out using “phage display,” a process well known to those skilled in the art. The aptamers were selected from a pool of recombinant bacteriophage expressing 10¹⁰ variants of a 15 amino acid long sequence based on ability to bind F_{c} in an ELISA pre-screen.

b. Synthesis and Characterization

The peptide aptamers selected in Example 2a are synthesized by standard peptide synthesis methodology. Aptamers to be further screened are modified to facilitate attachment to an array surface. A primary amine is positioned at the amino terminus of the aptamers and a 12 carbon alkyl spacer, designed to permit the aptamer to retain its essential three dimensional conformation and to allow orientation away from the underlying supporting substrate, is also included.

The aptamers are spotted onto a substrate that is prepared as follows. A 4×4 mm polished silicon chip is coated with 5 nm chromium followed by 30 nm of pure gold. The chip is then dipped in an alkanethiolate solution containing a C-16 alkane having a terminal succinimide group, followed by a 2 hour incubation and rinsing with ethanol. Next, aptamers are printed onto the surface by microjetting spots approximately 40 μm in diameter at indexed locations. The spontaneous coupling of the terminal succinimide group to the terminal amino group of the aptamers takes at 95% relative humidity for 2 hours, followed by rinsing. Free succinimide groups on the array surface are blocked with 10 mM glycine. The array is rinsed and used immediately without drying.

One μl of F_{c} protein (0.1 mg/ml) in phosphate buffered saline is added to the array. The array is incubated for 30 minutes, rinsed and placed into the AFM for imaging. The height of each domain is measured. Because the height of the aptamers immobilized in the domains is relatively small in comparison to the height of the F_{c} protein, the change in height for bound vs. unbound aptamers is easily measurable.

In subsequent steps, the binding conditions are varied and the experiment is repeated using the aptamer arrays described above. The degree of binding is monitored as a function of increasing salt concentration, temperature, and chaotropic reagent (urea, guanidine HCl) concentration. As the stringency of the binding conditions increases, a corresponding decrease in binding is observed in a subset of the domains. Ultimately, the most robust species (for the conditions tested) is identified.

### Example 3

### AFM Detection of Anti-HIV gp120 Binding to Viral Protein Nanoarray

Antibodies directed against specific proteins were characterized as follows. Immobilized recombinant Human Immunodeficiency Virus coat protein gp120 (HIV gp120) (Biodesign International, Saco, Me.) was bound with antibody and interrogated with AFM to reveal absolute levels of fidelity and cross-reactivity under a specific set of conditions.

Glass cover slips (#1) (Fisher Scientific, Pittsburgh, Pa.) were cut to 4 mm squares and cleaned by sonicating in 18 MΩ water for 15 minutes followed by sonicating in absolute ethanol for 15 minutes. The surfaces were blown dry under a stream of dry argon and sputter coated with 3 nm of chromium (99.99%) and 15 nm of gold (99.99%) using an ion beam sputterer (South Bay Technology, San Clemente, Calif.). An electron microscopy grid was used to mask the surface during sputtering. The gold-coated glass substrates were used immediately or stored in a clean environment at room temperature and used within 3-4 days.

Recombinant HIV gp120 (0.88 mg/ml) and purified polyclonal antibodies against HIV gp120 (3-4 mg/ml) were obtained from Biodesign International, Saco, Me. HIV gp120 samples were prepared using spin columns (Pierce Biochemicals, Milwaukee, Wis.) to replace the supporting buffer with buffer A (10 mM Tris-HCl, pH 7.4 and 10 mM NaCl). The proteins were aliquoted and stored at −20C.

A Nanoarrayer deposition tool (BioForce Nanosciences, Ames, Iowa) was used to create an array. Prior to loading, the deposition tool was treated by exposure to ultraviolet light and ozone in a TipCleaner device (BioForce Nanosciences, Inc., Ames, Iowa) for 15 minutes. To load the deposition tool, a 1 μl drop of HIV gp120 (prepared as described above) was first air dried on a glass cover slip. The deposition tool was then mounted onto a custom manufactured piezo-actuated cantilever (10 mm long) on the NanoArrayer and brought into proximity of the dried protein. The dried protein spot was hydrated by introducing moist air near the spot. Simultaneously, the cantilever was extended to bring the deposition tool into contact the protein droplet. Protein spontaneously wicked onto the hydrophilic deposition probe by capillary action. This process was controlled and terminated by stopping the flow of moist air, after which the protein sample remained on the deposition tool. The device thus loaded was used to deposit several spots of HIV gp120 in a 4×4 square array having domains of 1-2 μm in diameter on the gold-coated array substrates prepared as described above.

The arrayed surfaces were then incubated with 1 μl of the anti-HIV gp120 antibody (0.1 mg/ml) in PBS, pH 7.4 and 0.5% Tween 80 at room temperature for 2 hours in a humidified environment. Prior to AFM imaging, the array was washed in a gentle stream of 10 mM PBS, pH 7.4 for 5-10 sec, followed by rinsing in 18 MΩ water. The array was then blown dry under a steam of dry argon.

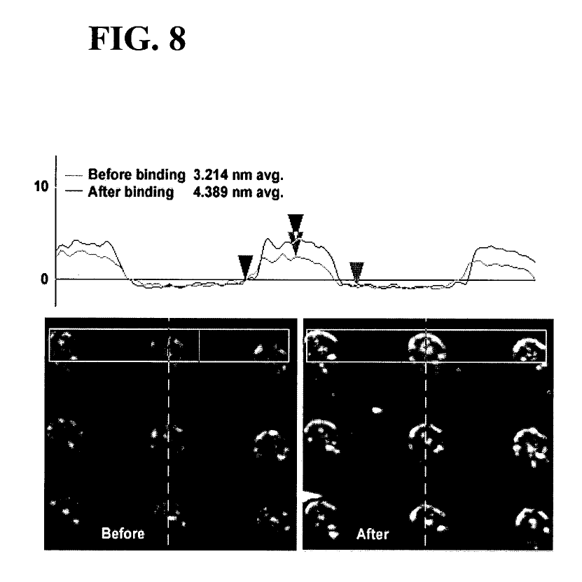

AFM imaging was performed in tapping mode on a Dimension 3100 (Digital Instruments/Veeco, Santa Barbara, Calif.) using non-contact ultralevers (Park Scientific Instruments, Santa Barbara, Calif.). Images were captured at a scan rate of 1 Hz with a resolution of 512×512 pixels. As shown in FIG. 8, the HIV gp120 antibody bound to the gp120 spots, resulting in an increase in the corresponding height profile of about 1 nanometer.

### Example 4

### AFM Detection of Virus Binding to Anti-Virus Antibody Nanoarray in the Presence of Serum Proteins

A 2×10 antibody array of mouse anti-CPV monoclonal antibody, mouse anti-CB3 (coxsackievirus B3) antibody and rabbit anti-bacteriophage fd (“anti-fd”) polyclonal antibody was prepared by microjetting 9 μm spots. Some domains were left blank as controls. The anti-fd/anti-CPV/anti-CB3 array was exposed to 1 μl of fd phage (10¹⁰ pfu/ml) in blocking buffer optimized for antibody-virus binding with minimal nonspecific binding for 30 minutes. AFM imaging revealed that an average of 35 fd particles were bound within each anti-fd domain. No fd particles were bound to the anti-CPV and anti-CB3 domains and to the antibody-free, background gold regions of the array.

Next, an identical array was exposed to 1 μl of CPV (60 μg/ml) in blocking buffer for 30 minutes. Upon AFM imaging, it was determined that approximately 250 CPV particles were bound in each 9 μm² anti-CPV domain. In contrast, an average of 4 CPV particles were associated with the fd and CB3 antibody domains. No CPV particles were found to bind on the background gold regions.

A third array was exposed to 1 μl of CB3 (5×10⁷ pfu/ml) in blocking buffer for 30 minutes. Upon AFM imaging, an average of 300 CB3 particles were bound to each 9 μm² anti-CB3 domain. On average, 2 CB3 particles were associated with the fd and CPV antibody domains.

To test the ability of this approach to function under typical biological conditions, the following experiments were performed. First, CPV, fd and CB3 in bovine serum was added to the antiviral array as described above. Upon AFM imaging, anti-CPV, anti-CB3 and anti-fd domains captured, on average, the same number of particles as when the experiment was performed in the absence of serum. Thus, the method was demonstrated to function in the presence of biologically relevant fluid.

Further experiments demonstrated that fd was bound when supplied in filtered culture media without a concentration step and that CB3 could be captured directly from both unpurified cell lysate and untreated sludge.

### Example 5

### Characterization of Optimal pH for Binding Immobilized Antibody to Bacteriophage fd Using Antibodies from Three Commercial Sources

Three different commercial antibody preparations (Fitzgerald, Sigma and Pharmacia) were tested for their ability to capture bacteriophage fd as imaged by AFM.

A 4×4 mm polished silicon substrate was coated with a pattern of metal by first coupling the silicon to a mask containing the desired pattern. In this experiment, an electron microscopy grid with a single 600 um diameter hole was used. An ion beam sputterer (South Bay Technology, San Clemente, Calif.) was used to deposit 5 nm of chromium as an adhesion layer, followed by 10 nm of 99.9999% gold. This surface was used within 4 days for deposition of antibodies.

Anti-fd antibodies in 50 mM phosphate buffer at pH 6.2, 6.8, and 7.4 and 50 mm Bicarbonate buffer at pH 8.3, 9.0 and 9.6 were patterned on the array by placing 1 microliter on the gold using a microjet device with a 30 um diameter orifice (Microfab Inc., Plano, Tex.). The antibodies were then allowed to spontaneously adsorb to the surface for 60 minutes, followed by rinsing with deionized water and used within 30 minutes.

Next, the array was incubated with μl of fd phage (10¹⁰ pfu/ml) in blocking buffer for 30 minutes.

AFM imaging was used to analyze the array. Five micron scan fields were collected in quintuplicate for each sample. The surface-bound fd particles in each scan field were counted by hand and the mean number of particles was calculated for each antibody under each condition. The results for antibodies from Sigma and Pharmacia are shown in Table 1.

TABLE 1
Average particle counts for anti-fd antibodies

| pH | Sigma | Pharmacia |
|---|---|---|
| 6.2 | 107 | 130 |
| 6.85 | 114 | 151 |
| 7.4 | 145 | 125 |
| 8.3 | 43 | 54 |
| 9.0 | 65 | 43 |
| 9.6 | 68 | 24 |

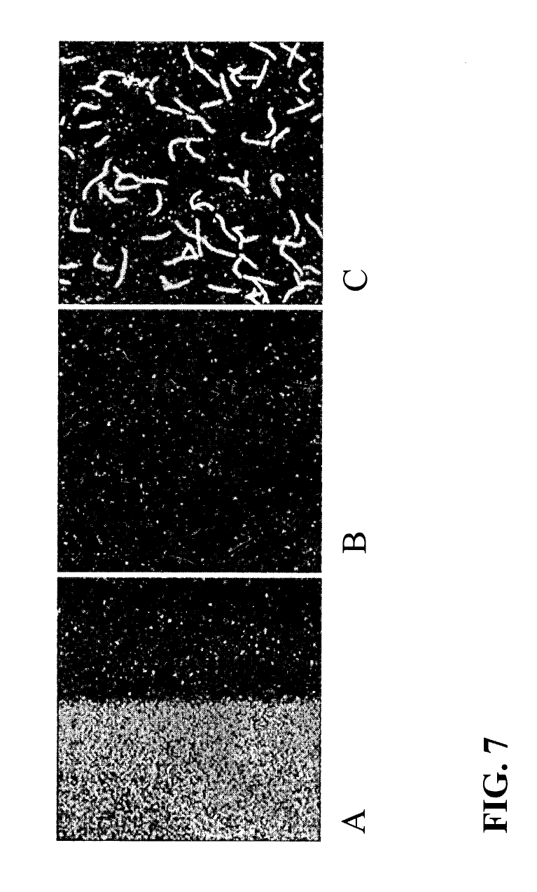

As shown in FIG. 7, the total particle counts determined from the AFM images clearly showed that the antibody obtained from Sigma Chemical Company (Panel C, 50 particles) was superior for binding bacteriophage fd in the surface immobilized assay format for pH 7.35. Antibodies obtained from Pharmacia (Panel B, 26 particles)) and Fitzgerald (Panel A, 0 particles) performed less well. Hence, for development of an anti-fd array of the type described herein, the Sigma antibody was demonstrated to be the best candidate. Moreover, the optimal pH for binding by all antibodies tested was approximately 7.35. Thus, for gold surfaces and a spontaneous immobilization method of attachment, the antibodies adsorbed from buffered solution in the range of about 7.0 to about 7.5 function to bind their target most efficiently.

### Example 6

### Selection of Substrate

In this experiment, varying treatments on glass, silicon and mica substrates are tested for ability to immobilize antibodies in an array format. In each case, a 4×4 mm piece of the substrate material is prepared.

First, test substrates are rinsed with acetone or ethanol, followed by a UV treatment generated by a mercury vapor bulb (wavelength about 180 nm to 400 nm) for 5 to 15 minutes. Each of the substrates are then treated as set forth in Table 2.

TABLE 2

| Substrate | Treatment 1 | Treatment 2 | Treatment 3 |
|---|---|---|---|
| Glass | None | 5 nm | Self-assembling |
| | | chromium | monolayer |
| | | followed by | |
| | | 20 nm gold | |
| Mica | None | 5 nm | Self-assembling |
| | | chromium | monolayer |
| | | followed by | |
| | | 20 nm gold | |
| Silcon | None | 5 nm | Self-assembling |
| | | chromium | monolayer |
| | | followed by | |
| | | 20 nm gold | |

Treatment 2 is carried out using an ion beam sputterer, resulting in a pure surface that is free from contamination until exposure to ambient conditions. The gold surfaces are used immediately after sputtering to minimize contamination from air borne oils and other contaminants that could detrimentally impact the antibody-binding step, described below.

Treatment 3 results in the coating of the substrate with a self assembling monolayer (SAM) containing a 16 carbon alkanes with succinimide at one end and a sulfhydryl group (SH) at the other. The sulfur spontaneously binds to the gold with high affinity and creates a surface with attachment chemistry.

The array is then coupled to antibodies by spontaneous adsorption or reactivity, depending on surface treatment. One microliter of antibody solution at a concentration of about 1 μg/μl is allowed to incubate with the surface for 30 minutes followed by rinsing with phosphate buffered saline. The surfaces thus prepared are used in a target binding assay with viral particles followed by surface imaging by AFM.

To ascertain the effect of the various treatments on antibody immobilization, the number of viral particles bound in each domain is determined and used as a measure of the functionality and density of antibodies coupled to the surfaces under each tested condition. The most appropriate substrate/surface treatment can then be used in assays addressing further research questions.

While the present invention has now been described and exemplified with some specificity, those skilled in the art will appreciate the various modifications, including variations, additions and omissions, that may be made in what has been described. Accordingly, it is intended that these modifications also be encompassed by the present invention and that the scope of the present invention be limited solely be the broadest interpretation that lawfully can be accorded the appended claims.

All patents, publications and references cited herein are hereby fully incorporated by reference. In case of conflict between the present disclosure and incorporated patents, publications and references, the present disclosure should control.

We claim:

1. A method of detecting a molecular interaction, comprising the steps of:
a) contacting an array with one or more target molecules, the array comprising a plurality of different affinity molecules in discrete domains, each domain having a predefined address in the array;
b) interrogating the array with a probe having a tip to create a profile of the array; and
c) evaluating the profile to detect an interaction between at least one affinity molecule and at least one target molecule.

2. The method of claim 1, wherein the probe is an atomic force microscope probe.

3. The method of claim 2, wherein the probe measures at least one physical property.

4. The method of claim 3, wherein the target molecules are delivered to the array in a liquid sample.

5. The method of claim 4, wherein the physical property is height, morphology, compliability, friction or viscoelasticity, or combinations thereof.

6. The method of claim 3, wherein the tip comprises one or more affinity molecules or target molecules.

7. The method of claim 6, wherein the physical property is friction, affinity, avidity, binding force, or rupture force, or combinations thereof.

8. The method of claim 1, wherein the affinity molecules comprise monoclonal antibodies or portions thereof.

9. The method of claim 1, wherein the affinity molecules comprise aptamers.

10. The method of claim 1, wherein the affinity molecules comprise antigens.

11. A method of determining antibody specificity comprising:
a) contacting an array with an antigen, the array comprising a plurality of antibodies arranged in discrete domains, each of the domains having a predefined address in the array;
b) interrogating the array with a probe having a tip to create a profile of the array;
c) evaluating the profile to detect an antibody-antigen interaction in one or more of the domains; and
d) correlating the antibody-antigen interaction with antibody specificity.

12. The method of claim 11, wherein the antigen is modified.

13. The method of claim 12, wherein the antigen is modified by binding with blocking antibodies of known specificity.

14. The method of claim 12, wherein the antigen is modified by deletion or substitution mutagenesis.

15. The method of claim 11, wherein the antibodies are monoclonal antibodies.

16. The method of claim 11, wherein the tip comprises one or more antibodies of known specificity.

17. A method of determining antibody specificity comprising:
a) contacting an array with at least one antibody, the array comprising a plurality of antigens arranged in discrete domains, each of the domains having a predefined address in the array;
b) interrogating the array with a probe having a tip to create a profile of the array;
c) evaluating the profile to detect an antibody-antigen interaction in one or more of the domains; and
d) correlating the antibody-antigen interaction with antibody specificity.

18. The method of claim 17, wherein the antigens are modified.

19. The method of claim 18, wherein the antigens are modified by binding with blocking antibodies of known specificity.

20. The method of claim 18, wherein the antigens are modified by deletion or substitution mutagenesis.

21. The method of claim 17, wherein the antibodies are monoclonal antibodies.

22. The method of claim 17, wherein the tip comprises one or more antibodies of known specificity.

23. A method of characterizing a molecular interaction comprising the steps of:
a) contacting an array with one or more target molecules under defined reaction parameters, the array comprising a plurality of affinity molecules in discrete domains, each domain having a predefined address in the array;
b) interrogating the array with a probe having a tip to create a profile of the array;
c) evaluating the profile to detect an interaction between at least one affinity molecule and at least one target molecule in one or more domains; and
d) correlating the interaction with the binding conditions to characterize the molecular interaction.

24. The method of claim 23, wherein the probe is an atomic force microscope probe.

25. The method of claim 24, wherein the probe measures at least one physical property in each of the domains.

26. The method of claim 24, wherein the tip comprises an affinity molecule or a target molecule.

27. The method of claim 25, wherein the physical property is friction, compliability, height, morphology, viscoelasticity, rupture force, binding force, affinity or avidity, or combinations thereof.

28. The method of claim 23 wherein the reaction parameters are selected from the group consisting of tonicity, temperature, pH, humidity, pressure, or combinations thereof.

29. A method of selecting a substrate for an array of immobilized molecules comprising:
a) contacting an array with at least one target molecule, the array comprising a plurality of substrates arranged in discrete domains and at least one affinity molecule disposed on the substrates in each of the domains:
b) interrogating the array with a probe having a tip to create a profile of the array;
c) evaluating the profile to detect a molecular interaction in one or more of the domains; and
d) selecting one or more of the substrates based on the profile.

30. The method of claim 29, wherein the probe is an atomic force microscope probe.

31. The method of claim 30, wherein the probe measures at least one physical property in each of the domains.

32. The method of claim 31 wherein the physical property is friction, compliability, height, morphology, viscoelasticity, rupture force, binding force, affinity or avidity, or combinations thereof.

33. The method of claim 29, wherein the tip comprises an affinity molecule or a target molecule.

34. A method of determining target occupancy time comprising:
a) contacting an array with one or more target molecules, the array comprising a plurality of affinity molecules in discrete domains, each domain having a predefined address in the array;
b) interrogating the array with a probe having a tip to detect onset of binding between at least one target molecule and at least one affinity molecule;
c) interrogating the array of step b) with a probe having a tip to detect dissociation of at least one target molecule and at least one affinity molecule: and
d) measuring the time between onset of binding detected in step c) and dissociation detected in step c) to determine target occupancy time.
